(19) United States
(12) Patent Application Publication
(10) Pub. No.: US 2023/0380418 A1
(43) Pub. Date: Nov. 30, 2023

(54) COMPOSITION, AGROCHEMICAL FORMULATION, METHODS FOR INCREASING WATER AND NUTRIENT AVAILABILITY AND FOR IMPROVING PEST CONTROL IN PLANTS AND SEEDS, AND THE AGROCHEMICAL FORMULATION

(71) Applicant: Oxiteno S.A. Indústria e Comércio, São Paulo (BR)

(72) Inventors: Rafael PIRES-OLIVEIRA, Mauá (BR); Poliana Cardoso GUSTAVSON, Mauá (BR); Bruna MENDONÇA, Mauá (BR)

(73) Assignee: Oxiteno S.A. Indústria e Comércio, São Paulo, SP (BR)

(21) Appl. No.: 18/032,577

(22) PCT Filed: Oct. 15, 2021
(86) PCT No.: PCT/BR2021/050449

§ 371 Date: Apr. 19, 2023

(30) Foreign Application Priority Data
Oct. 19, 2020 (BR) BR102020021357-1
Apr. 7, 2021 (BR) BR102021006626-1

(51) Int. Cl. A01N 25/04; A01P 5/00; A01P 13/00; A01N 43/80; A01N 43/653; A01N 37/22; A01N 35/06; A01N 43/70; A01N 43/90

(52) U.S. Cl. CPC A01N 25/04; A01P 5/00; A01P 13/00; A01N 43/80; A01N 43/653; A01N 37/22; A01N 35/06; A01N 43/70; A01N 43/90

(57) ABSTRACT

The present invention relates to a composition comprising ethoxylated polyols, an agrochemical formulation comprising said composition, and the use thereof in agriculture. Additionally, the present invention also describes methods for increasing water and nutrient availability and for improving pest control in plants and seeds.

### FIELD OF THE INVENTION

The present invention relates to a composition comprising ethoxylated polyols, an agrochemical formulation comprising said composition, and the use thereof in agriculture.

Additionally, the present invention also describes methods for increasing water and nutrient availability and for improving pest control in plants and seeds.

### BACKGROUND OF THE INVENTION

Water is an essential natural resource for agriculture, where it is used in crop irrigation. It is estimated that agriculture is responsible for the consumption of about 70% of available water resources.

With deficient irrigation, plants are subjected to hydric stress, leading to a reduction in their growth and productivity. Furthermore, under stress, plants are more susceptible to pest and disease attacks, also negatively affecting their productivity.

Additionally, water is a means of transport, as well as being a solvent for various compounds in the soil, such as electrolytes, natural signaling compounds, nutrients, pesticides (e.g. herbicides, insecticides, nematicides, and fungicides), among others, besides being essential for the living microorganisms that inhabit the soil.

Soil supports plants' roots, providing a foundation for their growth and supplying nutrients. Its natural constitution is quite heterogeneous and is basically composed of four elements: minerals, organic matter (including microorganisms), water and air in the pores (Weil and Brady (2017) The Nature and Properties of Soils, 15^{a} ed., Pearson Education). The structure, mineral composition, and amount of organic matter are factors that affect the interaction of soil and water, which can affect whether the soil is repellent to water.

The characteristic of soils that are poorly wettable is called water repellency or hydrophobicity and can be classified by the penetration time of water droplets (Maia et al. (2005) Identifição de Repelência à Água em Solos sob Plantios Florestais. Colombo: Embrapa Florestas (Comunicação técnica 147)).

Plants release an exudate through the root that consists of polysaccharides. After a period of drought, this exudate forms a hydrophobic film that also makes it difficult for the plant to absorb water after rehydration of the soil (Zarebanadkouki et al. (2018) Plant Soil, 428, 265-277).

Therefore, irrigation of crops in areas where the soil displays water repellency behavior, as it does not allow water penetration, or displays low water retention capacity resulting in a low availability of water and nutrients for the plants, becomes even more challenging in regions where water is scarce.

In addition, in regions where access to water is restricted, there is an additional need for pumping the water from the ground, which represents an extra energy cost for this operation.

To overcome these problems that may cause water stress on plants, resulting in a reduced yields and increased susceptibility to disease and pest attack, many products containing surfactants have been developed, and are commercialized, to be applied in areas from turf to extensive, irrigated agricultural crops.

Thus, the main products on the market to solve the problem of water repellent soils comprise the use of surfactants. The majority of these products use surfactants based on oxyalkylene block copolymer chemistry, typically poly(ethylene oxide)-poly(propylene oxide)-poly(ethylene oxide) copolymers (PEO-PPO-PEO) or abbreviated EO/PO. These compounds are commercially known as Poloxamers and the ULTRARIC® (OXITENO) or Pluronic® trademarks and have varied block or randomized repetitions of the ethylene oxide units (EO) and propylene oxide (PO).

The surfactant property of EO/PO mixed copolymers has long been well known in the literature (Alexandridis et al. (1994) Langmuir, 10, 2604-2612) and, for this reason, it has been the object to constitute different products.

For instance, U.S. Pat. No. 6,851,219 B2 reports a process to increase hydrophilicity and water infiltration into a water repellent soil. The process is based on the soil application of a wetting agent comprising a blend of EO/PO block copolymer and alkyl polyglucoside (APG). The increase in soil wettability rate is due to the synergistic effect of the mixture composition in the 6:1 to 0.5:1 mass ratio of APG relative to the EO/PO copolymer. The inventors show that, as expected, the higher the molecular weight of the hydrophobic fraction, the faster the water penetrates the soil. For this reason, the inventors state that one EO/PO copolymer suitable for this application would be with a molecular weight of 2,000 to 8,000, hydrophilic fraction of 10% to 40%, and HLB value less than or equal to 10, together with APG containing 4 to 22 carbons in the alkyl chain and a degree of polymerization of 1 to 4.

U.S. Pat. No. 7,541,386 B2 describes that the mixture component APG has the function of increasing the cloud point (CP) of the mixture, making possible the solubilization in water of previously water insoluble components. For this reason, the inventors use, in water, an EO/PO copolymer with HLB value less than or equal to 2, molecular weight greater than 3,000, and hydrophilic fraction less than or equal to 10% with the effect of improving water infiltration into hydrophobic soils.

Thus, the focus of these two patents is on the use of mixed EO/PO block copolymer with different compositions in order to reduce the surface tension of the aqueous solution and, consequently, reduce the penetration time of water into the soil. This feature of EO/PO copolymers has also been explored in other patents with the same objective, such as: U.S. Pat. Nos. 5,595,957 A and 6,857,225 B2, EP 2811829 B1, BR 112019016058 A2, WO 2019/057617 A1, and U.S. Pat. No. 10,196,567 B2.

Document BR 112019016058 A2 concerns a method to reduce water repellency in soil and/or to increase water infiltration in soil comprising the use of an EO/PO mixed copolymer blend in blocks (tri and/or pentablocks) with an alkoxylated alcohol derived from 2-propyl heptanol and/or an ethoxylated alcohol derived from isotridecanol. The results show that the application of different blend compositions of these components can increase soil water infiltration and reduce soil water repellency.

Other molecules can also be modified to have a surfactant behavior and consequently improve the penetration of water into the soil. U.S. Pat. No. 10,352,011 B2, for instance, refers to a wetting composition for increasing soil moisture retention with a compound derived from mono-, di-, or trifunctional alcohols (naphthol, diethylene glycol, and glycerol are shown in the examples, respectively) with both EO and PO groups, in block or randomly, with a molecular weight from 2,000 to 6,000 g/mol, being a suitable compound the trifunctional compound (glycerol) rich in the PO group.

In EP 3294790 B1, the same inventors perform the same type of derivatization of di- and trifunctional alcohols, obtaining products with molecular weights from 1,000 to 6,000 g/mol and HLB value from 2 to 6, aiming to increase the water infiltration rate. In this case, the best results shown were achieved using PO-rich glycerol or comprising lower EO content. The molecules shown in the examples in the European patent exhibit amphiphilic character and, from the HLB value, it can be concluded that they are rich in the PO group.

U.S. Pat. No. 6,857,225 B2 describes a sandy soil additive formulation for turf comprising glycerol and/or sorbitol, both alkoxylated with at least one PO group, with the function of being a wetting agent (amphiphilic). This composition reduces the surface tension of the aqueous solution and reduces dry spots in the treated area. The inventors demonstrate that the hexa-ramified compound (alkoxylated sorbitol) performs better than the tri-ramified compound (alkoxylated glycerol) as a wetting agent.

Document WO 2019/057617 A1 refers to a formulation to increase water retention in the soil composed of an EO/PO alcohol; a surfactant selected from the group of sulfosuccinates, alkyl sulfosuccinates, ethoxylated alcohol, EO/PO copolymers, APG, and combinations thereof; and a humectant selected from the group of propylene glycol, butylene glycol, pentylene glycol, hexylene glycol, triethylene glycol, tripropylene glycol, polyethylene glycol, polypropylene glycol, glycerol, sorbitol, xylitol, mannitol, lactic acid, diacetin, triacetin, and combinations thereof, wherein such wetting components are non-alkoxylated or derived.

Thus, it can be observed that the state of the art, in general, uses mixed EO/PO block copolymers with low HLB value, or uses APG or alkoxylated alcohol in its composition to enhance the water infiltration result in water repellent soils.

It has been found, however, that such compounds have low water retention capacity in soils and/or make use of higher chain alkoxylated derivatives of branched polyols to reduce the surface tension value, such as PO (propylene oxide) or a mixed EO/PO alkoxylation.

In this manner, it was surprisingly observed in the present invention that a composition comprising ethoxylated polyols with lower molecular weight, rather than a mixed and/or higher chain alkoxylation, shows excellent results in increasing water and nutrient availability for plants and seeds, in addition to providing better pest control.

Unexpectedly, it was found in this invention that the composition comprising ethoxylated polyols can even be used in agriculture at lower concentrations, such as at least 10 times lower than the concentrations of compositions normally employed.

In addition, the ethoxylation process has the advantage of being simpler than a combined process of ethoxylation and propoxylation, and the final product displays very distinct physicochemical properties.

### SUMMARY OF THE INVENTION

The present invention relates to a composition comprising a compound of general formula (I)

(R¹—O—R)m (I)

where m is a number ranging from 3 to 6,

R¹ is a C₁ alkyl radical,

each R is independently H or an oxyethylene group represented by [(C₂H₄O)n-R²) with the proviso that at least one R is [(C₂H₄O)n-R²],

R² is independently a hydrogen or a C₁₋₄ alkyl chain,

each n can be the same or different and is a number ranging from 1 to 18, and the sum of all n present in the compound of formula (I) is a number that ranges from 1 to 108.

The present invention also relates to an agrochemical formulation comprising said composition which comprises the compound of formula (I) and at least one active ingredient with pesticidal or plant enhancing action.

A third objective of the present invention is to provide a method for increasing the availability of water and nutrients to plants and seeds comprising

providing a composition comprising a compound of general formula (I)

(R¹—O—R)m (I)

where m is a number ranging from 3 to 6,

R¹ is a C₁ alkyl radical,

each R is independently H or an oxyethylene group represented by [(C₂H₄O)n-R²) with the proviso that at least one R is [(C₂H₄O)n-R²],

R² is independently a hydrogen or a C₁₋₄ alkyl chain,

each n can be the same or different and is a number ranging from 1 to 18, and the sum of all n present in the compound of formula (I) is a number that ranges from 1 to 108; and

applying the composition to a seed, soil, liquid medium, or inert substrate.

Further, the present invention also provides a second method for improving pest control in plants and seeds comprising

supplying the agrochemical formulation comprising the compound of formula (I) and at least one active ingredient with pesticidal or plant enhancing action; and

applying the agrochemical formulation to a seed, soil, liquid medium, or inert substrate.

A final objective of the present invention relates to uses of agrochemical composition and formulation in agriculture.

### BRIEF DESCRIPTION OF THE FIGURES

FIG. 1 shows water retention curves in sandy loam soil, lot 1.

FIG. 2 shows water retention curves in sandy loam soil, lot 2.

FIG. 3 shows the results of the infiltration test of the examples in water repellent soil.

### DETAILED DESCRIPTION OF THE INVENTION

The present invention describes a composition comprising a compound belonging to the group of ethoxylated polyols. The compound has the general formula (I)

(R¹—O—R)m (I)

where m is a number ranging from 3 to 6,

R¹ is a C₁ alkyl radical,

each R is independently H or an oxyethylene group represented by [(C₂H₄O)n-R²) with the proviso that at least one R is [(C₂H₄O)n-R²],

R² is independently a hydrogen or a C₁₋₄ alkyl chain,

each n can be the same or different and is a number ranging from 1 to 18, and the sum of all n present in the compound of formula (I) is a number that ranges from 1 to 108.

Thus, in the present invention, the compound of formula (I) is a polyol derivative having covalent bonds between the R¹ radicals, and the majority molecular weight of the oxyethylene groups in the compound of formula (I) can vary from 44 g/mol to 4,752 g/mol.

The composition of the present invention comprises from about 5% to about 100% by weight of ethoxylated polyol of general formula (I) based on the total weight of the composition.

In alternative embodiments, the composition may comprise from about 5% to about 80% by weight of the general formula compound (I) or from about 8% to about 60% of the compound of general formula (I) based on the total weight of the composition.

In one embodiment of the present invention, the compound of general formula (I) is a polyol derivative with m being 3 and at least one R being [(C₂H₄O)n-R²], wherein R² is hydrogen, each n is independently a number between 1 and 18 and the sum of all n present in the compound of formula (I) is a number that ranges from 1 to 50. Thus, in these realizations, the majority molecular weight of the oxyethylene groups in the compound with formula (I) ranges from 44 g/mol to 2,200 g/mol.

In a particular embodiment, the compound of general formula (I) is a polyol derivative with m being 3 and all R being [(C₂H₄O)n-R²], wherein R² is hydrogen, each n is independently a number between 1 and 18 and the sum of all n present in the compound of formula (I) is a number ranging from 3 to 50. Therefore, in these realizations, the majority molecular weight of the oxyethylene groups in the compound with formula (I) ranges from 132 g/mol to 2,200 g/mol.

In certain instances, the compound of general formula (I) is a polyol derivative with m being 3 and all R being [(C₂H₄O)n-R²], wherein R² is hydrogen, each n is independently a number between 1 and 18 and the sum of all n present in the compound of formula (I) is a number that ranges from 7 to 28. Therefore, in these realizations, the majority molecular weight of the oxyethylene groups in the compound with formula (I) ranges from 308 g/mol to 1,232 g/mol.

In an alternative embodiment, the compound of general formula (I) is a polyol derivative with m being 6, at least one R being [(C₂H₄O)n-R²], where R² is hydrogen, each n is independently a number between 1 and 18 and the sum of all n present in the compound of formula (I) is a number ranging from 6 to 60. Therefore, in these configurations, the majority molecular weight of the oxyethylene groups in the compound of formula (I) ranges from 264 g/mol to 2,640 g/mol.

Alternatively, the compound of general formula (I) is a polyol derivative with m being 6 and all R being [(C₂H₄O)n-R²], where R² is hydrogen, each n is independently a number between 1 and 18 and the sum of all n present in the compound of formula (I) is a number that ranges from 6 to 54. Therefore, in these configurations, the majority molecular weight of the oxyethylene groups in the compound of formula (I) ranges from 264 g/mol to 2,376 g/mol.

In the embodiment above, the compound of general formula (I) is a polyol derivative with m being 6 and all R being [(C₂H₄O)n-R²], where R² is hydrogen, each n is independently a number between 1 and 18 and the sum of all n present in the compound of formula (I) is a number ranging from 30 to 50, although other compounds may be employed. Therefore, in these configurations, the majority molecular weight of the oxyethylene groups in the compound of formula (I) ranges from about 1,320 g/mol to 2,200 g/mol.

In this invention, the term “majority” and its derivatives are used because of the molecular weight variations that are inherent from the ethoxylation process, wherein the final product exhibits a distribution of chain sizes. Therefore, in the context of the present invention, “majority” is to be understood as the maximum point of oxyethylene groups in the chain size distribution of the compound of formula (I).

Examples of polyols that can be used in the present invention are polyols containing 3 to 6 carbons and 3 to 6 reactive hydroxyls, such as 1,2,3-Propanetriol and 1,2,3,4,5,6-Hexanehexol. Thus, after ethoxylation reaction, i.e., reaction with ethylene oxide, the resulting molecule may contain polymer chains comprising from 1 to 18 oxyethylene groups, and up to 108 oxyethylene groups per mole of polyol.

The composition of the present invention may also comprise one or more additional components, such as surfactants, unmodified polyols, water, dyes, preservatives, defoamers, freeze inhibitors, and their like, among others.

In an alternative embodiment, the composition described herein additionally comprises from about 0% to about 60% by weight of at least one surfactant, from about 0% to about 40% by weight of at least one unmodified polyol, and water in q.s. (“quantum satis”, which means the amount which is enough for) 100% by weight, based on the total weight of the composition.

Alternatively, the composition additionally comprises from about 0% to about 45% by weight of at least one surfactant, from about 0% to about 30% by weight of at least one unmodified polyol, and water in q.s. (“quantum satis”, which means the amount which is enough for) 100% by weight, based on the total weight of the composition.

In an alternative embodiment, the composition may comprise from about 5% to about 45% by weight of at least one surfactant, from about 0% to about 10% by weight of at least one unmodified polyol, and water in q.s. (“quantum satis”, which means the amount which is enough for) 100% by weight, based on the total weight of the composition.

Other additional components such as colorants, preservatives, defoamers, and freeze inhibitors may be present in the composition in a range from 0% to 60% by weight, based on the total weight of the composition.

The surfactants, if present in the composition described, can be selected from one or more of the group comprising ethoxylated alkyl ethers, phosphated ethoxylated alkyl ethers, ethoxylated alkyl etheramines, alkyl polyglycosides, ethoxylated alkyl polyglycosides, ethoxylated imidazolines, polysiloxane derivatives, alkyl dimethyl amine oxides, alkyl dimethyl betaines, trialkyl ammonium propanoates, alkyl amido propyl amines, ethoxylated alkyl amines, ethoxylated amidoamines, alkylene oxide block or random copolymer, sorbitan esters, polysorbates or the like.

The unmodified polyols, if present in the composition of the present invention, may be selected from one or more of the group comprising 1,2,3-Propanetriol, 1,2,3,4-Butanetetrol, 1,2,3,4,5-Pentanepentol, and 1,2,3,4,5,6-Hexanehexol.

The present invention also relates to agrochemical formulations comprising the composition containing the ethoxylated polyol of general formula (I) and at least one active ingredient. An active ingredient is understood to be a component with pesticidal or plant-improving action, which can be selected from the group comprising, but not limited to, herbicides, insecticides, fungicides, nematicides, or the like.

In the embodiment in which at least one active ingredient is a herbicide, the classification according to the Herbicide Resistance Action Committee is used as a definition (HRAC) in relation to chemical classes, namely: acetamides, arylaminopropionic acid, benzoic acid, chlorocarbonic acid, phenoxycarboxylic acid, phosphinic acid, quinolinecarboxylic acid, amides, aryloxyphenoxypropionates (FOPs), arylpicolinate, benzamides, benzofurans, benzothiadiazinones, bipyridyliums, carbamates, cyclohexanediones (DIMs), chloroacetamides (V1), chloroacetamides (V2), chloroacetamides (V3), diphenyl ethers, dinitroanilines, dinitrophenols, phenylcarbamates, phenylpyrazoles, phenylpyrazolines (DENs), phenyl-pyridazines, phosphoroamidates, phosphorodithioates, semicarbazone phthalamates, glycines, imidazolinones, long chain fatty acid inhibitor, isoxazoles, isoxazolidinones, N-phenylphthalimides, nitriles, organoarsenicals, oxadiazoles, oxazolidinedinones, oxiacetamides, pyrazoles, pyrazoliums, pyridazinones, pyridines, pyridinecarboxamides, pyrimidindiones, pyrimidinyl (thio) benzoates, sulfonylaminocarbonyl-triazolinones, sulfonylureas, tetrazolinones, thiadiazoles, thiocarbamates, triazines, triazinones, triazoles, triazolinones, triazolocarboxamides, triazolopyrimidines, tricetones, uracils, ureas or the like.

In the embodiment in which at least one active ingredient is an insecticide, it is used as the definition of the classification according to the Insecticide Resistance Action Committee (IRAC) in relation to chemical classes, namely: group of METI acaricides and insecticides, acequinocyl, aliphatic halogenated, amitraz, nereistoxin analogues, juvenile hormone analogues, avermectins, milbemicins, azadirachtin, Bacillus sp. and the insecticidal proteins that they produce, benzoylurea, benzoximate, bifenazate, borates, borax, bromopropylate, buprofezin, butenolides, lime sulphur, carbamates, carboxanilides, cyanides, cyclodienes, cyromazine, clofentezine, diflovidazin, hexithiazoxy, chlorfenapyr, dinitrophenol, sulfluramid, chloropicrin, DDT, methoxychlor, tetronic and tetramic acid derivatives, betaketonitrile derivatives, azomethine pyridine derivatives, diacylhydrazines, diafenthiuron, diamides, dicofol, spinosynes, ethoxazole, phenylpyrazoles (fiproles), fenoxycarb, rotenoid-type saponin flavones, flonicamide, fluacripyrim, fluorides, phosphides, methyl isothiocyanate generators, hydramethylnone, inorganic, inorganic phosphine precursor, methyl isothiocyanate precursor, organophosphate, mesoionics, neonicotinoids, nicotine, organotins, organophosphates, oxadiazines, GS-omega/kappa HXTX-Hvla peptide, pyrazole, pyrethroids and pyrethrins, pyridalyl, pyriproxyfen, propargite, quinomethionate, rotenone, semicarbazones, fluoroaliphatic sulfonamide, sulfoxaflor, tetradifone, or the like.

In the embodiment in which at least one active ingredient is a fungicide, we use the definition of the classification according to the Committee for Resistance Action to Fungicides (FRAC) in relation to chemical classes, namely: 1,2,4-thiadiazole, 2,6-dinitro-aniline, 4-quinolyl-acetate, carboxylic acid, enopyranuronic acid, phthalamic acid, acylalanine, allylamine, mandelic acid amides, cinnamic acid amides, aminocyanoacrylate, amino-pyrazolinone, anilinopyrimidine, anthraquinone, aryloxyquinoline, Bacillus sp. and the produced fungicide lipopeptides, benzene sulfonamide, benzylcarbamate, benzimidazole, benzisothiazole, benzophenone, benzoylpyridine, benzothiadiazole BTH, benzotriazine, butyrolactone, carbamate, carboxamide, cyanoacetamide-oxime, cyanoimidazole, cyano-methylene-thiazolidine, cyclopropane carboxamide, chloronitrile, triphenyltin compounds, dicarboximide, dihydro-dioxazine, dinitrophenyl crotonate, dithiocarbamates and relative, dithiolane, spirocetal-amine, ethyl phosphonate, ethylamino-thiazol-carboxamide, phenyl-acetamide, phenyl-benzamide, phenyl-oxo-ethyl thiophene amide, phenylpyrrole, phenylurea, phosphonate, phosphorothiolate, phthalimide, furan-carboxamide, guanidine, aromatic hydrocarbon, terpene hydrocarbons and terpene alcohols, hydroxy-(2-amino-) pyrimidine, hydroxyaniline, imidazole, imidazolinone, inorganic (copper), inorganic (sulfur), isobenzofuranone, isothiazolone, isoxazole, maleimide, methoxy-acetamide, methoxy-acrylate, methoxy-carbamate, Reynoutria sp. extract, morpholine, N-phenyl carbamate, N-methoxy-(phenyl-ethyl)-pyrazol-carboxamide, pyrimidine peptidyl nucleoside, oxatin-carboxamide, oxazolidine-dione, oxazolidinone, oximine-acetamide, oximine-acetate, piperazine, piperidine, piperidinyl-thiazole-isoxazoline, pyrazole-4-carboxamide, pyrazole-5-carboxamide, pyridazinone, pyridine, pyridine-carboxamide, pyridinyl-ethyl benzamide, pyridinylmethyl-benzamide, pyrimidine, pyrimidinamine, pyrimidinone-hydrazone, pyrrolo-quinolinone, polypeptide (lectin), polysaccharide, propionamide, quinazolinone, quinoxaline, sulfamoyl-triazole, sulfonamide, tetrazoyloyloxime, thiadiazol-carboxamide, thiazol-carboxamide, thiocarbamate, thiophanate, thiophene-carboxamide, toluamide, triazine, triazole, triazolintione, triazolobenzo-thiazole, triazole-pyrimidylamine, Trichoderma sp. and produced fungicidal metabolites, trifluoroethylcarbamate, valinamide carbamate or the like.

In the embodiment in which the at least one active ingredient is a nematicide, an ingredient comprising at least one of the following chemical classes is considered a nematicide: halogenated aliphatic, avermectin, benzamides, plant extract, fluoroalkenyle (-thiother), methyl isothiocyanate precursor, benzofuranyl methylcarbamate, organophosphate or the like.

In one embodiment of the present invention, the agrochemical formulation further comprises at least one additional ingredient selected from nutrients, microorganisms, and growth regulating/biostimulant compounds.

Both macronutrients and micronutrients can be used as a nutrient. The macronutrients can be selected from one or more of N, P, K, S, Ca, Mg, or the like, while the micronutrients can be selected from one or more of Fe, Zn, Mn, Cu, Ni, Cl, Mo, B, Si, Se, Al, Co, V, Na, or the like.

Bacteria, fungi, mites, nematodes, among others, such as Amblyseius sp., Azozpirillum sp., Bacillus sp., Baculovirus sp., Beauveria sp., Cotesia sp., Cryptolaemus sp., Deladenus sp., Heterorhabditis sp., Isaria sp., Metarhizium sp., Neoseiulus sp., Orius sp., Paecilomyces sp., Pasteuria sp., Phytoseiulus sp., Pseudomonas sp., Stratiolaelaps sp., Telenomus sp., Trichoderma sp., Trichogramma sp., or the like may be present as microorganisms in the agrochemical formulation.

The growth regulating/biostimulant compounds that may be present in the agrochemical formulation of the present invention comprise one or more of the following compounds: dioxocyclohexanecarboxylic acid, indolalcanoic acid, aliphatic alcohol, quaternary ammonium, carbimide, carboxanilide, cycloalkene, cyclohexadione, cytokinin, dinitroaniline, ethylene inhibitor, ethylene precursor, gibberellin, pyridazinadione, sesquiterpenes, triazole, benzothiadiazol, nitric oxide, methyl salicylate, methyl jasmonate, proteins, polypeptides, polyamines, algae extracts, fulvic acid, humic acid, plant growth promoting rhizobacteria, or the like.

The present invention also relates to a method for increasing the availability of water and nutrients for plants and seeds, wherein the method comprises the following steps:

providing a composition comprising a compound of general formula (I)

(R¹—O—R)m (I)

where m is a number ranging from 3 to 6,

R¹ is a C₁ alkyl radical,

each R is independently H or an oxyethylene group represented by [(C₂H₄O)n-R²) with the proviso that at least one R is [(C₂H₄O)n-R²],

R² is independently a hydrogen or a C₁₋₄ alkyl chain,

each n can be the same or different and is a number ranging from 1 to 18, and the sum of all n present in the compound of formula (I) is a number that ranges from 1 to 108; and

applying the composition to a seed, soil, liquid medium, or inert substrate.

The application step of the composition can be directly by seed treatment or by gravity irrigation system, by sprinkling, by dripping, by infiltration, by spraying, by central pivot irrigation, by hydroponics system, or by submergence. The composition can be applied in a concentration ranging from about 1 ppm to 10,000 ppm.

It is possible to apply the composition of the present invention in different areas such as agricultural plantations, ornamental lawn/turf areas, forests, plant growth substrates, large irrigated agricultural crops such as corn, soybeans, cotton, wheat, rice, tomatoes, peanuts, garlic, onions, fruits (banana, watermelon, melon, citrus, strawberry, grapes, blueberry, raspberry), fresh vegetables (lettuce, chicory, arugula, spinach, watercress), vegetables (cassava, potato, beans, fava beans, pumpkin, pepper, beet, radish, cucumber), flowers (sunflower, rose, carnation, tulip, marigold, daisy), or the like, among others.

The plant growth substrate may be any inert substrate, organic or mineral, that facilitates the anchoring of plants in hydroponic systems, i.e., growing without soil. Non-limiting examples include peat, sand, gravel, mineral wool, synthetic foams, expanded clay, and vermiculite, among others.

The present invention also describes an alternative method for improving pest control in plants and seeds, wherein the method comprises the steps of:

providing the agrochemical formulation described here; and

applying the agrochemical formulation to the seed, soil, liquid medium, or inert substrate.

As with the previously described method, the application step of the agrochemical formulation can be directly by seed treatment or by gravity irrigation system, sprinkler, drip, or infiltration, by spraying, by central pivot irrigation, by hydroponic system, or by submergence. The formulation can be applied in a concentration ranging from about 1 ppm to 10,000 ppm.

Likewise, the agrochemical formulation can be applied in any area, such as those described herein.

The present invention also relates to the use of the agrochemical composition or formulation described herein in agriculture.

In one embodiment, the use is for application to the soil, liquid medium, or inert substrate to increase the availability of water and nutrients to plants and seeds or to improve pest control in them, the areas and possible means of application being as described herein.

The main advantages obtained by this invention include reduced stress caused by water deficit, improved mobility of compounds to plants and seeds, such as pesticides, nutrients, and biostimulant compounds, as well as enhancing the development of microorganisms of interest.

Thus, the methods and uses described in the present invention promote increased availability of water, nutrients, and pesticides to plants and seeds, the water being from natural precipitation or irrigation. Consequently, for example, the infiltration time of water into the repellent soil/substrate is reduced and the mobility of components and nutrients to the plants in the areas of interest is facilitated.

Consequently, one advantage of the methods and uses described herein is the productivity of plants of relevance to agricultural crops. Productivity proved impressively higher when the agrochemical compound/formulation was applied even under deficit irrigation conditions, resulting in the production of more fruit that was larger in diameter.

Moreover, it was observed that the germination rate of seeds was unexpectedly improved when they were kept moistened with the agrochemical composition/formulation of the present invention as compared to using pure water.

It was also possible to observe that the agrochemical formulation, when applied to the soil, surprisingly promoted an increase in pest control, as it provided an increase in the efficiency of active ingredients with pesticidal action, in addition to an improvement in the maintenance of the viability of these organisms after 1 year of storage.

The invention is best described based on the examples given below.

### EXAMPLES

### Example 1: Water Retention Curve

The water retention curve of soils characteristic of tropical regions was evaluated using the compositions detailed in Table 1.

In these realizations, the compound of general formula (I) used was ethoxylated 1,2,3-propanetriol with a majority of 26 oxyethylene groups, equivalent to the majority molecular weight of oxyethylene groups in the polyol of 1,144 g/mol.

In certain compositions, the following was/were used as additional component(s): an unmodified polyol (1,2,3,4,5,6-Hexanehexol), and/or surfactants (isodecyl alcohol 3 EO, isodecyl alcohol 6 EO, lauric alcohol 7 EO, alkyl polyglucoside, polysorbate 20, Poloxamers 182 and Poloxamers 334).

TABLE 1
Compositions 1 to 10, wherein compositions 1, 5, 8, 9, and 10 are those that represent
the present invention, while the rest are compositions according to the prior art.

| | Composition (% by weight) | | | | | | | | | |
|---|---|---|---|---|---|---|---|---|---|---|
| Component | 1 | 2 | 3 | 4 | 5 | 6 | 7 | 8 | 9 | 10 |
| Ethoxylated | 10 | | | | 10 | | | 21 | 21 | 21 |
| polyol | | | | | | | | | | |
| Isodecyl | 3.5 | | | | | | | | | |
| alcohol 3 EO | | | | | | | | | | |
| Isodecyl | | | | | | | 10 | 20.9 | 20.9 | |
| alcohol 6 EO | | | | | | | | | | |
| Lauric alcohol | | | | | | | | | | 20.9 |
| 7 EO | | | | | | | | | | |
| Alkyl | 7 | 14 | 14 | 14 | | | | | | |
| polyglucoside | | | | | | | | | | |
| Polysorbate 20 | | 10 | | | | | | | | |
| Acrylic | | | | 10 | | | | | | |
| polymer | | | | | | | | | | |
| (comparative | | | | | | | | | | |
| water retainer) | | | | | | | | | | |
| Poloxamers 182 | | | | | | | | | 20.9 | |
| Poloxamers 334 | | | 10 | | | 10 | | 20.9 | | 20.9 |
| Unmodified | | | | | 3.5 | 3.5 | 3.5 | 7.4 | 7.4 | 7.4 |
| polyol | | | | | | | | | | |
| Water | q.s. | q.s. | q.s. | q.s. | q.s. | q.s. | q.s. | q.s. | q.s. | q.s. |
| | 100% | 100% | 100% | 100% | 100% | 100% | 100% | 100% | 100% | 100% |

The water retention characterization was performed on soil characteristic of tropical regions, described as a sandy loam soil (˜20 to 25% clay), being a less hydrophilic soil than clayey soils. Deformed samples were collected from diagnostic horizons of this soil to perform the tests.

These samples were air dried and sieved on 2 mm mesh sieves (Terra Fina Seca ao Ar—TFSA). The soils were packed into 100 cm³ volumetric rings and saturated with a mixture of water and a sample of tested compound.

In comparison, the test was performed with a commercially available formulation for this application, Watermaxx2® (sample called “Benchmark”) and water as a control. The “Benchmark” formulation comprises 10% of mixed EO/PO alkoxylated polyols, 7% of alkyl polyglucoside and the balance in water.

The samples of the compounds were diluted with 10 parts water for this experiment. All treatments were performed in triplicate.

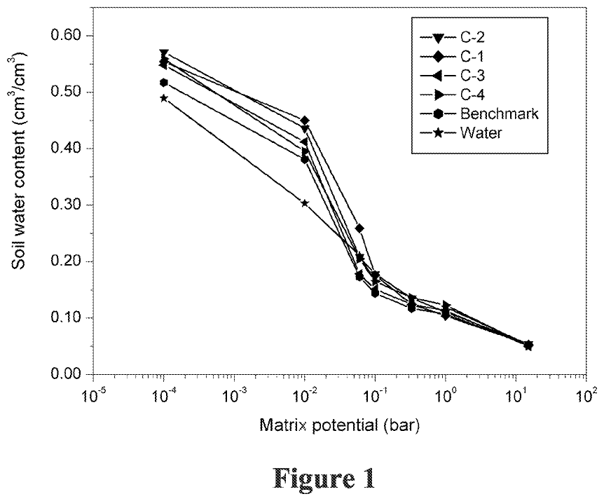

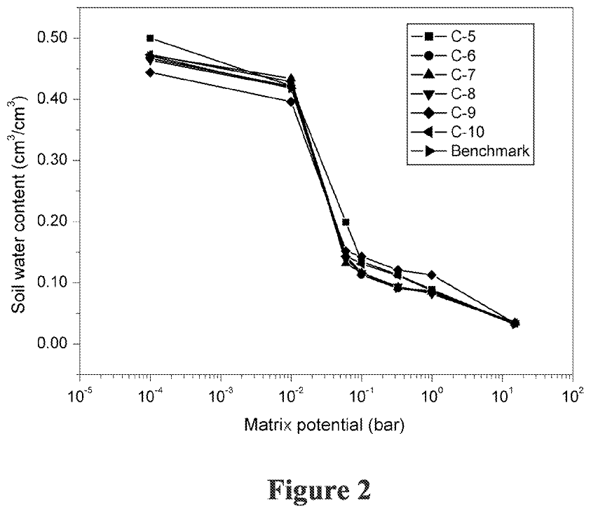

Water retention was determined by means of the soil water retention characteristic curve using a tension table (Topp and Zebchuk (1979) Canadian Journal of Soil Science, 59, 19-26) and Richards' Chambers (Klute (1986) Methods of soil analysis: Part 1—Physical and mineralogical methods. 2nd ed. Madison: SSSA. chap. 26). After the drainage ceased, the water retained in the soil was quantified, the volumetric moisture content was calculated, and the curves in FIGS. 1 and 2 were constructed.

Using the soil water retention curve data, relevant soil physical and hydraulic figures for plant development were calculated. The total porosity (maximum retentive capacity of the soil), macroporosity, microporosity, plant-available water content (AWC), and the water storage capacity per hectare were determined for both soils.

The calculation of soil macroporosity resulted from the difference between the saturated soil volumetric moisture and the volumetric moisture at field capacity, which, in this work, was considered the volumetric moisture determined at a matrix potential of −0.6 bar (−60 kPa).

Total porosity is equivalent to saturated soil bulk moisture, and microporosity is equivalent to bulk moisture at field capacity (FC). The calculation of AWC (Tables 2 and 3) for plants resulted from the difference between the moisture content at field capacity (−0.6 bar) and the moisture at the permanent wilting point, which corresponds to the moisture measured at a matrix potential of −15 bar (−1,500 kPa).

The Water Storage Capacity (WSC) per hectare refers to the amount of water (in cubic meters, m³) that can be stored in one hectare of soil and is available to plants up to a depth of one meter and it is calculated using the following equation:

$$W S C = \frac{( A W C \times {10}^{7} )}{1000}$$

where:

WSC is the water storage capacity in m³/ha and

AWC is the available water content in cm³/cm³.

To complement the information of available water content for plants, the water storage in cubic meters was calculated (m³) per hectare (ha). This data represents the amount of water that this soil would make available to plants up to a depth of one meter after gravitational water drainage.

The data was submitted to variance analysis in an entirely random design, and the means were compared using Tukey's test, with (p<0.05).

TABLE 2
Values of the soil's physical and hydraulic indicators of
the different treatments calculated based on the parameters
of the water retention curve of sandy loam soil, lot 1. Results
with different letters are significantly different.

| | Available Water | |
|---|---|---|
| Sample | Content (cm³/cm³) | WSC (m³/ha) |
| Composition 1 | 0.20 a | 2,003.06 a |
| Composition 2 | 0.15 abc | 1,523.58 abc |
| Composition 3 | 0.12 bc | 1,232.23 bc |
| Composition 4 | 0.15 abc | 1,522.93 abc |
| Benchmark | 0.13 bc | 1,269.57 bc |
| Water (control) | 0.12 bc | 1,221.25 bc |

The data in Table 2 show a higher plant-available water content and water storage capacity when the soil is treated with composition 1, which comprises the compound of general formula (I) that increases the water retention in the soil.

TABLE 3
Values of the soil's physical and hydraulic indicators of
the different treatments calculated based on the parameters
of the water retention curve of sandy loam soil, lot 2. Results
with different letters are significantly different.

| | Available Water | |
|---|---|---|
| Sample | (cm³/cm³) | WSC (m³/ha) |
| Composition 5 | 0.17 a | 1 661.14 a |
| Composition 6 | 0.11 bc | 1 084.37 bc |
| Composition 7 | 0.10 c | 974.04 c |
| Composition 8 | 0.11 bc | 1 109.01 bc |
| Composition 9 | 0.10 bc | 1 047.71 bc |
| Composition 10 | 0.12 b | 1 180.88 b |
| Benchmark | 0.11 bc | 1 097.55 bc |

The data in Table 3 show, surprisingly, a greater content of plant-available water and water storage capacity, with statistical difference, when the soil is treated with the composition containing the compound of general formula (I).

Composition 5 showed the best result for available water content and water storage capacity. Compositions 8, 9, and 10 also showed very satisfactory and surprisingly superior results in comparison to composition 7, which is free of ethoxylated polyols and comprises only a surfactant and an unmodified polyol.

### Example 2: Tomato Yield

The agronomic efficacy of the composition containing the ethoxylated polyol of general formula (I) of the present invention, according to the compositions described in Table 4.

Again, the compound of general formula (I) used was ethoxylated 1,2,3-Propanetriol with a majority of 26 oxyethylene groups, equivalent to the majority molecular weight of oxyethylene groups in the polyol of 1,144 g/mol. In all compositions, an unmodified polyol (1,2,3,4,5,6-Hexanehexol) and surfactants selected from Isodecyl Alcohol 6 EO, Lauryl Alcohol 7 EO, Poloxamers 182 and Poloxamers 334, or mixtures thereof, were used as additional components.

TABLE 4
Compositions 11 to 13 containing a compound
with the general formula (I).

| | Composition (% by weight) | | |
|---|---|---|---|
| Component | 11 | 12 | 13 |
| Ethoxylated polyol | 21.0 | 11.9 | 19.4 |
| Isodecyl alcohol 6 EO | 21.0 | 11.9 | |
| Lauric alcohol 7 EO | | | 19.3 |
| Poloxamers 182 | | 12.0 | |
| Poloxamers 334 | 21.0 | | 19.6 |
| Unmodified Polyol | 7.4 | 4.2 | 6.8 |
| Water | q.s. 100% | q.s. 100% | q.s. 100% |

To conduct the agronomic efficacy study of the formulations containing the ethoxylated polyol of the present invention, presented in Table 4, an experiment was conducted in a greenhouse according to an experimental design described in the literature (Tang et al. (2017) Scientific Reports, 7, 10009).

The objective of the study was to evaluate tomato yield and quality in plants subjected to hydric stress and treated with compositions described in the present invention. The experiment was carried out in an entirely randomized design, with 4 replicates. In the study, each experimental unit corresponded to a pot with a volume of 3.6 L with a single tomato seedling planted. The field capacity (FC) of the soil was determined experimentally, and the pots were irrigated with 500 ppm solutions of the compositions in Table 4, at different regimes of 100% and 50% FC in order to impose hydric stress on the plant. Field capacity is understood to be the amount of water available in the soil to be used by the plant.

In comparison, the test was performed with the commercially available formulation for this application Watermaxx2® (sample called “Benchmark” as described in Example 1) and water as a control. After the 8^{th}, 10^{th}, and 14^{th} weeks, the ripe tomatoes were harvested measuring their weight on a precision scale and their size with a caliper. The results are shown in Table 5.

TABLE 5
Tomato yield treated with the compositions 12 and 13 containing
the compound general formula (I) under deficit irrigation.

| | | | | Number | Number | Number | | Average |
|---|---|---|---|---|---|---|---|---|
| | | Total | | of | of | of | Total | size |
| | Irrigation | volume | Total | fruits | fruits | fruits | weight | of the |
| | Regime | applied | number | 8^{th} | 10^{th} | 14^{th} | harvested | fruits |
| | (%) | (L) | fruit | week | week | week | (g) | (mm) |
| Composition | 50% FC | 4.635 | 11 | 5 | 1 | 5 | 28.3 | 17.5 |
| 12 | | | | | | | | |
| Composition | 50% FC | 4.262 | 6 | 3 | 1 | 2 | 15.0 | 16.2 |
| 13 | | | | | | | | |
| Benchmark | 50% FC | 4.600 | 4 | 2 | 0 | 2 | 9.7 | 16.1 |
| Water | 50% FC | 4.527 | 3 | 0 | 0 | 3 | 5.8 | 14.9 |
| Water | 100% FC | 7.218 | 6 | 1 | 5 | 0 | 13.9 | 15.8 |

The data in Table 5 show that tomatoes treated with the compositions comprising ethoxylated polyol of general formula (I) surprisingly produced a larger amount of, and/or larger, ripe fruit at the first harvest in the 8^{th} week.

The results obtained with Compositions 12 and 13 show that the plant, besides not responding negatively to water stress even when irrigated at 50% FC, still had a significantly higher fruit yield when compared to the plant irrigated at 50% FC with only water or with the Benchmark and even higher than the plant irrigated with water even at 100% FC.

The application of compositions comprising ethoxylated polyol of general formula (I) enables the volume reduction of water used for crop irrigation, without negatively affecting the productivity of the plant. Thus, water, a scarce natural resource, can be saved and used more efficiently by the plant.

### Example 3: Water Droplet Penetration Time (WDPT) Method

To evaluate water infiltration in water repellent soil, experiments were conducted as described in the literature (Maia et al. (2005) Identifiçãgdo de Repêlencia à Água em Solos sob Plantios Florestais. Colombo: Embrapa Florestas (Comunicação técnica 147)).

Briefly, commercially available garden soil samples that exhibit water repellency were sieved on a 1,000 μm mesh sieve. The samples were then arranged in Petri dishes and dried in an oven at 60° C. for 24 hours; cooling in a desiccator after this period. When they reached room temperature, the soils were leveled, and a drop of 40 μL of 0.05 wt % aqueous solution of the compositions described in Table 4 was deposited on the soil surface, compared to the commercially available formulation for this application Watermaxx2® (sample called “Benchmark”, as described in example 1) and water as a control, and the time for the droplet to infiltrate the soil was measured.

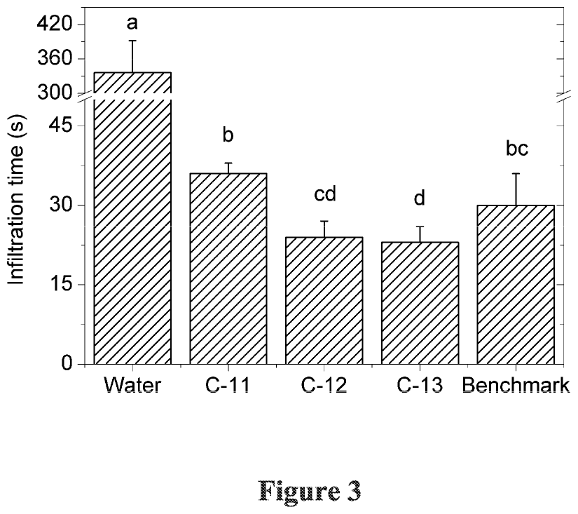

As shown in FIG. 3, the results obtained were submitted to the variance analysis in an entirely random design, and the means were compared by the Tukey test, with (p<0.05).

The results are shown in FIG. 3, where one can see that the compositions comprising the compound of general formula (I) significantly reduced the infiltration time of the water in the repelling soil.

### Example 4: Germination Rate

The influence on germination of the compositions described in the present invention was also evaluated, according to Table 6.

The compounds of general formula (I) used were: ethoxylated 1,2,3-Propanetriol with a majority of 26 oxyethylene groups, equivalent to the majority molecular weight of oxyethylene groups in the polyol of 1,144 g/mol, and ethoxylated 1,2,3,4,5,6-Hexanehexol with a majority of 40 oxyethylene groups, equivalent to the majority molecular weight of oxyethylene groups in the polyol of 1,760 g/mol.

In certain compositions, the unmodified polyol 1,2,3,4,5,6-Hexanehexol was used as an additional component.

TABLE 6
Compositions 14-25.

| | Composition (% by weight) | | | | | | | | | | | |
|---|---|---|---|---|---|---|---|---|---|---|---|---|
| Component | 14 | 15 | 16 | 17 | 18 | 19 | 20 | 21 | 22 | 23 | 24 | 25 |
| 1,2,3- | 30 | 40 | 50 | 30 | 30 | 30 | | | | | | |
| Propanetriol | | | | | | | | | | | | |
| ethoxylate | | | | | | | | | | | | |
| 1,2,3,4,5,6 | | | | | | | 30 | 40 | 50 | 30 | 30 | 30 |
| Hexanehexol | | | | | | | | | | | | |
| ethoxylate | | | | | | | | | | | | |
| Unmodified | | | | 5 | 10 | 15 | | | | 5 | 10 | 15 |
| Polyol | | | | | | | | | | | | |
| Water | q.s. | q.s. | q.s. | q.s. | q.s. | q.s. | q.s. | q.s. | q.s. | q.s. | q.s. | q.s. |
| | 100% | 100% | 100% | 100% | 100% | 100% | 100% | 100% | 100% | 100% | 100% | 100% |

The germination test was conducted in order to evaluate if the compositions could negatively interfere in this very sensitive phase of development of seeds. For this test and following assessment protocols (Toledo and Marcos Filho (1977) Manual das sementes: tecnologia da produção, São Paulo: Agronômica Ceres), 100 seeds of different crops were placed on a germination paper previously moistened with 20 mL of the aqueous solution to be tested (Table 6).

The seeds were covered with another germination paper, moistened again with 20 mL of the solution, and the papers were rolled up, stored in a closed transparent plastic bag, and kept in a climate-controlled Conviron plant growth chamber for 15 days (27° C., photoperiod 16 h). After this period, the number of germinated seeds was counted (Table 7 below) and the characteristics of the radicle were visually evaluated. The results show that the compositions comprising the compounds of general formula (I) do not impair the germination rate of lettuce and tomato seeds and, notably, root size even increased, as well as being more uniform, when compared to the control sample.

TABLE 7
Germination rate of seeds treated with compositions
of the general formula compound (I).

| Sample | Lettuce | Tomato |
|---|---|---|
| Water (control) | 81 | 97 |
| Composition 14 | 87 | 94 |
| Composition 15 | 88 | 95 |
| Composition 16 | 93 | 98 |
| Composition 17 | 100 | 95 |
| Composition 18 | 66 | 95 |
| Composition 19 | 98 | 94 |
| Composition 20 | 93 | 91 |
| Composition 21 | 99 | 98 |
| Composition 22 | 84 | 95 |
| Composition 23 | 78 | 90 |
| Composition 24 | 80 | 92 |
| Composition 25 | 94 | 94 |

### Example 5: Nematode Control Boost

The efficacy of the agrochemical formulation of the present invention in nematicide treatments was evaluated, as shown in Table 8 below.

For this realization, the composition comprising the compound of formula (I) contained in the agrochemical formulation comprises about 47% by weight of ethoxylated 1,2,3-Propanetriol with a majority of 26 oxyethylene groups, equivalent to the majority molecular weight of oxyethylene groups in polyol of 1,144 g/mol, about 23% unmodified polyol (1,2,3,4,5,6-Hexanehexol), and the balance in water.

The determination of the synergistic action of the agrochemical formulation containing the compound of general formula (I) on increasing soil water retention with gall nematode control products (Meloidogyne javanica) was conducted in the greenhouse using soybeans (Glycine max cv. Don Mario 5958i), which is susceptible to Meloidogyne javanica.

One thousand (1,000) juveniles and eggs of Meloidogyne javanica were inoculated and the following commercially available and widely known nematicide products were used: Ilevo® (active ingredient: Fluopyram), Quartzo® (active ingredient: Bacillus subtilis and B. lichemformis), Votivo Prime® (active ingredient: Bacillus firmus) and Rizotec® (active ingredient: Pochonia chlamydosporia), with or without the compound of formula I, according to Table 8.

The formulations were applied via seed treatment (0.600 L/100 kg seed) or sowing furrow (50 L/ha), according to the indication of each product. A randomized block experimental design was carried out, with five repetitions each treatment, with one plant per pot.

TABLE 8
Nematicide treatments applied to soybean inoculated
with the Meloidogyne javanica.

| | Dosage (L, | |
|---|---|---|
| | kg/100 kg | |
| Treatments | seeds) | Application |
| Non-inoculated control | — | — |
| Inoculated control | — | — |
| Composition comprising | 0.50 | Seed treatment |
| compound of formula I | | |
| Votivo Prime ® | 0.10 | Seed treatment |
| Votivo Prime ® + composition | 0.10 + 0.50 | Seed treatment |
| comprising the compound of | | |
| formula I | | |
| Ilevo ® | 0.50 | Furrow |
| Ilevo ® + composition comprising | 0.50 + 0.50 | Furrow |
| the compound of formula I | | |
| Ilevo ® (2/5 of the dose) | 0.20 | Furrow |
| Ilevo ® (2/5 of the dose) + | 0.20 + 0.50 | Furrow |
| composition comprising the | | |
| compound of formula I | | |
| Quartzo ® | 0.20 | Furrow |
| Quartzo ® + composition | 0.20 + 0.50 | Furrow |
| comprising the compound of | | |
| formula I | | |
| Rizotec ® | 0.25 | Furrow |
| Rizotec ® + composition | 0.25 + 0.50 | Furrow |
| comprising the compound of | | |
| formula I | | |

The following parameters were evaluated: phytotoxicity at 7 and 14 days after emergence (DAE) in the form of a percentage of damage to the plants of the plot; plant emergence at 7 days after sowing (DAS) by counting the number of plants in 1 linear; plant vigor at 7 and 14 DAE by assigning a score of 100% vigor to the untreated control and by comparison assigning scores above this for possible increases in size, coloration, or below for possible phytotoxic effects; plant height at 30 and 60 DAE using a ruler graduated in millimeters (height was considered as the distance between the ground and the active growing point of the plant); and the number of nematodes in the soil and root at 30 and 60 DAE by the procedure detailed below.

The methodology for nematode extraction followed standard procedures available in the literature (Boneti and Ferraz (1981) Fitopatologia Brasileira, 6, 553; Coolen and D'Herde (1972) A Method for the Quantitative Extraction ofNematodesfrom Plant Tissue. Ghent: State Agricultural Research Centre; Jenkins (1964) Plant Disease Reporter, 48, 692).

Briefly, from each sample a 100 cm³ aliquot of soil was taken and the roots were separated for further extraction. The separated soil was placed in a bucket containing 4 L of water, and this soil was mixed with water until it becomes homogeneous. This solution was passed through a 20 mesh sieve (850 mm), the liquid was collected in a second bucket, the solution was rested for 30-45 seconds to decant, and then the collected liquid in the bucket was passed through a 400 mesh sieve (0.038 mm opening).

The roots from each sample were separated, washed, and dried with paper towels to be weighed. They were then chopped to the approximate size of 1.0 cm. To compose a 5.0 g sample, the samples were homogenized and ground in a blender containing 250 mL of water twice at 30 s intervals. With the soil and root samples prepared, 3.0 g of kaolin was added (analytical composite) to the soil samples and 6.0 g to the root samples.

The material was transferred to centrifuge tubes for two centrifugation steps (1,800 RPM for 5 min; 1,800 RPM for 1 min). The supernatant was discarded, and the deposit was suspended in a sucrose-based solution (400 g sugar in 750 mL water).

Then, 50 to 75 mL of this sucrose solution was added to cuvettes and homogenized until all the sedimented material get released from the bottom of each cuvette. At the end of the second cycle, the contents of each cuvette were poured onto a 500 mesh sieve (0.025 mm opening) over a column of water after a few seconds until the liquid became colorless, the material retained on the sieve was transferred to “snap cap” bottles. Around 50 to 80 mL was collected. After the process, the flasks were placed in a water bath (54° C., lethal temperature), and 1 mL of formaldehyde (50%) was added in each sample.

For counting and identification, the samples, reduced to 10 mL after decantation, were taken under a stereomicroscope (2 mL of each sample), the number of nematodes being counted in duplicate using a Peters chamber. The result obtained for the calculation of nematodes per 100 cm³ of soil and 5 g of roots. Calculation for soil: Reading 1+Reading 2=X*10=Total nematodes in 100 cm³ of soil. Calculation for root: Reading 1+Reading 2=X*10=Total nematodes in 5 g root. When the root weight is less than 5.0 g it is calculated: (Reading 1+Reading 2=((X*10)*5)/Weight of root). For the calculation of the percentages of efficiency resulting from the action of the tested nematicides, we used ABBOTT's formula (Abbott (1925) Journal of Economic Entomology, 18, 265-267):

$$Control efficiency ( \% ) = ( \frac{T - N}{T} ) \times 10$$

where:

T=number of nematodes in the control group

N=number of nematodes in the treatment.

The values for the number of nematodes and eggs sampled (x) have been transformed to √(x+1.0). These data and the others were submitted to variance analysis and the means were compared using the Scott Knott test at 5% probability. The statistical package AgroEstat Version 1 was used in the data analysis. The results are presented in Tables 9, 10 and 11.

TABLE 9
Number of Meloidogyne javanica nematodes in soybean
roots with different treatments at 30 DAE.

| | Juveniles | | Eggs | |
|---|---|---|---|---|
| Treatment | Averages | Eff. (%) | Averages | Eff. (%) |
| Non-inoculated control | 0 d | 0.0 | 0 c | 0.0 |
| Inoculated control | 409 a | 0.0 | 275 a | 0.0 |
| Composition comprising | 349 a | 14.7 | 282 a | 0.0 |
| compound of formula I | | | | |
| Votivo Prime ® | 178 b | 56.4 | 118 b | 57.0 |
| Votivo Prime ® + | 148 b | 63.8 | 83 b | 69.8 |
| Composition comprising | | | | |
| the compound of formula I | | | | |
| Ilevo ® | 74 c | 81.8 | 29 c | 89.3 |
| Ilevo ® + composition | 38 c | 90.7 | 19 c | 93.1 |
| comprising the compound of | | | | |
| formula I | | | | |
| Ilevo ® (2/5 of the dose) | 79 c | 80.6 | 97 b | 64.7 |
| Ilevo ® (2/5 of the dose) + | 41 c | 90.1 | 17 c | 93.8 |
| composition comprising the | | | | |
| compound of formula I | | | | |
| Quartzo ® | 187 b | 54.3 | 104 b | 62.1 |
| Quartzo ® + composition | 161 b | 60.6 | 111 b | 59.7 |
| comprising the compound of | | | | |
| formula I | | | | |
| Rizotec ® | 152 b | 62.8 | 112 b | 59.2 |
| Rizotec ® + composition | 134 b | 67.3 | 82 b | 70.0 |
| comprising the compound of | | | | |
| formula I | | | | |

Averages followed by different letters in the column are significantly different by the Scott-Knott test (p < 0.05).

TABLE 10
Number of Meloidogyne javanica nematodes in soybean
soil with different treatments at 60 DAE.

| | Juveniles | | Eggs | |
|---|---|---|---|---|
| Treatment | Averages | Eff. (%) | Averages | Eff. (%) |
| Non-inoculated control | 0 c | 0.0 | 0 b | 0.0 |
| Inoculated control | 234 a | 0.0 | 48 a | 0.0 |
| Composition comprising | 232 a | 0.9 | 48 a | 0.0 |
| compound of formula I | | | | |
| Votivo Prime ® | 210 a | 10.3 | 44 a | 8.3 |
| Votivo Prime ® + | 112 b | 52.1 | 32 a | 33.3 |
| composition comprising | | | | |
| the compound of formula I | | | | |
| Ilevo ® | 104 b | 55.6 | 22 a | 54.2 |
| Ilevo ® + composition | 40 c | 82.9 | 8 b | 83.3 |
| comprising the compound of | | | | |
| formula I | | | | |
| Ilevo ® (2/5 of the dose) | 168 a | 28.2 | 18 a | 62.5 |
| Ilevo ® (2/5 of the dose) + | 60 c | 74.4 | 10 b | 79.2 |
| composition comprising the | | | | |
| compound of formula I | | | | |
| Quartzo ® | 158 a | 32.5 | 24 a | 50.0 |
| Quartzo ® + composition | 144 a | 38.5 | 36 a | 25.0 |
| comprising the compound of | | | | |
| formula I | | | | |
| Rizotec ® | 182 a | 22.2 | 28 a | 41.7 |
| Rizotec ® + composition | 214 a | 8.5 | 16 b | 66.7 |
| comprising the compound of | | | | |
| formula I | | | | |

Averages followed by different letters in the column are significantly different by the Scott-Knott test (p < 0.05).

TABLE 11
Number of Meloidogyne javanica nematodes in soybean
roots with different treatments at 60 DAE.

| | Juveniles | | Eggs | |
|---|---|---|---|---|
| Treatment | Averages | Eff. (%) | Averages | Eff. (%) |
| Non-inoculated control | 0 c | 0.0 | 0 e | 0.0 |
| Inoculated control | 7816 a | 0.0 | 12617 b | 0.0 |
| Composition comprising | 11974 a | 0.0 | 14618 a | 0.0 |
| compound of formula I | | | | |
| Votivo Prime ® | 5084 b | 34.9 | 6807 c | 46.0 |
| Votivo Prime ® + | 6494 b | 16.9 | 8539 c | 32.3 |
| composition comprising | | | | |
| the compound of formula I | | | | |
| Ilevo ® | 3140 c | 59.8 | 2657 e | 78.9 |
| Ilevo ® + composition | 1823 c | 76.7 | 2132 e | 83.1 |
| comprising the compound of | | | | |
| formula I | | | | |
| Ilevo ® (2/5 of the dose) | 3102 c | 60.3 | 3254 e | 74.2 |
| Ilevo ® (2/5 of the dose) + | 1459 c | 81.3 | 2051 e | 83.7 |
| composition comprising the | | | | |
| compound of formula I | | | | |
| Quartzo ® | 6243 b | 20.1 | 11016 b | 12.7 |
| Quartzo ® + composition | 4193 b | 46.3 | 4864 d | 61.4 |
| comprising the compound of | | | | |
| formula I | | | | |
| Rizotec ® | 6015 b | 23.0 | 7776 c | 38.4 |
| Rizotec ® + composition | 3097 c | 60.4 | 5124 d | 59.4 |
| comprising the compound of | | | | |
| formula I | | | | |

Averages followed by the same letter in the column do not differ by the Scott Knott test (p < 0.05).

Nematode infestation in the soil and roots reached an adequate level to discriminate between treatments in terms of their control efficacy. No phytotoxicity symptoms were observed in soy plants by the application of active ingredients associated with the composition containing the general formula compound (I) in seed treatment and/or via furrow.

In general, one notes that, surprisingly, the control of the Meloidogyne javanica nematode has been significantly improved with the agrochemical formulation of the present invention comprising the compound of formula (I) and commercial nematicides.

Thus, it was possible to demonstrate a synergistic effect with all these products, at their respective doses, and expressively increasing their efficiency, whether comprising chemical or biological active ingredient.

Moreover, in the case of the chemical active ingredient fluopyram, the association of the commercial product with the formulated compound (I) has enabled an extraordinary reduction of its dose to at least ⅖ of the original dose, without reducing its control efficiency.

In the case of the association with the biological actives, Bacillus firmus, Pochonia chlamydosporia, and the combination of the microorganisms Bacillus subtilis and B. lichemformis, the use of the agrochemical formulation of the present invention has led to a substantial increase in the control of the Meloidogyne javanica nematode by these active ingredients.

### Example 6: Weed Control

The efficacy of the agrochemical formulation of the present invention in pre-emergent herbicide treatments was evaluated, as shown in Table 12 below.

Thus, in this realization, a composition comprising about 47 wt % of ethoxylated 1,2,3-Propanetriol with a majority of 26 oxyethylene groups was used, equivalent to the majority molecular weight of oxyethylene groups in the polyol of 1,144 g/mol, about 23% unmodified polyol (1,2,3,4,5,6-Hexanehexol) and the balance in water.

The determination of the synergistic action of the agrochemical formulation comprising the compound of general formula (I) in increasing soil water retention with weed control products was conducted in open field, in the municipality of Patrocínio Paulista, SP, geographical coordinates 20° 50′56.7″ S; 47° 14′23.7″ W, 781 m asl. The seed bank in the area is dominated by monocotyledons, such as Digitaria insularis, Eleusine indica and Brachiaria plantaginea, in addition to broadleaves such as Bidens sp. and Ipomoea acuminate.

TABLE 12
Herbicide treatments and doses applied.

| No. | Treatments | Dosage (mL or g p.c. ha⁻¹) | | Active Ingredient |
|---|---|---|---|---|
| 1 | Control | — | | — |
| 2 | Yamato ® | 200 | | Pyroxasulfone |
| 3 | Yamato ® + composition | 200 + 0.1% | v/v* | Pyroxasulfone |
| | comprising the compound of | | | |
| | formula I | | | |
| 4 | Yamato ® + composition | 200 + 0.2% | v/v | Pyroxasulfone |
| | comprising the compound of | | | |
| | formula I | | | |
| 5 | Boral ® | 400 | | Sulfentrazone |
| 6 | Boral ® + composition | 400 + 0.1% | v/v | Sulfentrazone |
| | comprising the compound of | | | |
| | formula I | | | |
| 7 | Boral ® + composition | 400 + 0.2% | v/v | Sulfentrazone |
| | comprising the compound of | | | |
| | formula I | | | |
| 8 | Dual Gold ® | 1,200 | | Metolachlor |
| 9 | Dual Gold ® + composition | 1,200 + 0.1% | v/v | Metolachlor |
| | comprising the compound of | | | |
| | formula I | | | |
| 10 | Dual Gold ® + composition | 1,200 + 0.2% | v/v | Metolachlor |
| | comprising the compound of | | | |
| | formula I | | | |
| 11 | Callisto ® | 400 | | Mesotrione |
| 12 | Callisto ® + composition | 400 + 0.1% | v/v | Mesotrione |
| | comprising the compound of | | | |
| | formula I | | | |
| 13 | Callisto ® + composition | 400 + 0.2% | v/v | Mesotrione |
| | comprising the compound of | | | |
| | formula I | | | |
| 14 | Proof ® | 5,000 | | Atrazine |
| 15 | Proof ® + composition | 5,000 + 0.1% | v/v | Atrazine |
| | comprising the compound of | | | |
| | formula I | | | |
| 16 | Proof ® + composition | 5,000 + 0.2% | v/v | Atrazine |
| | comprising the compound of | | | |
| | formula I | | | |
| 17 | Spider ® | 35 | | Diclosulam |
| 18 | Spider ® + composition | 35 + 0.1% | v/v | Diclosulam |
| | comprising the compound of | | | |
| | formula I | | | |
| 19 | Spider ® + composition | 35 + 0.2% | v/v | Diclosulam |
| | comprising the compound of | | | |
| | formula I | | | |

% v/v in relation to the tank volume

The experiment was conducted in randomized block design, with 28 treatments and four repetitions. Each plot was 3.0 m wide by 10.0 m long (30.0 m²). The data obtained were submitted to variance analysis, and the mean of the treatments were compared using the Scott-Knott test at 5% probability.

The treatments were applied using a CO₂-based constant pressure sprayer with a bar with six nozzles spaced 0.5 m apart. Twin jet spray nozzle TJ06 11002 and tank volume of 160 L ha⁻¹ were used. At the application moment, the soil was freshly prepared, and there were no weeds present, so the effects were observed on the soil seed bank.

The control efficiency of Digitaria insularis plants was evaluated at 7, 14, 21, 28, 35, 42, 49, and 56 days after the treatments were applied. The evaluations were made using a scale of 0 to 100, where 0 (zero) corresponds to no demonstrated injury and 100 (one hundred) to plant death (Velini et al. (1995) Procedimentos para instalação, avaliação e análise de experimentos com herbicidas. Londrina: SBCPD.). The results are presented in Table 13.

TABLE 13
Control efficiency (%) of the weed complex by different pre-emergent herbicides associated
with different dosages of adjuvant at 7, 14, 21, 28, 35, 42, 49, and 56 days after
the application of treatments. Means followed by different letters in the column
are significantly different by the Scott-Knott test at 5% probability.

| Treatment | Days After Application (DAA) | | | | | | | | | | | | | | | |
|---|---|---|---|---|---|---|---|---|---|---|---|---|---|---|---|---|
| No. | 7 | | 14 | | 21 | | 28 | | 35 | | 42 | | 49 | | 56 | |
| 1 | 0 | B | 0 | B | 0 | C | 0 | C | 0 | D | 0 | E | 0 | E | 0 | F |
| 2 | 100 | A | 100 | A | 100 | A | 100 | A | 100 | A | 100 | A | 87 | A | 72 | B |
| 3 | 100 | A | 100 | A | 100 | A | 100 | A | 100 | A | 100 | A | 90 | A | 80 | B |
| 4 | 100 | A | 100 | A | 100 | A | 100 | A | 100 | A | 100 | A | 93 | A | 88 | A |
| 5 | 100 | A | 100 | A | 94 | A | 88 | A | 85 | B | 81 | B | 75 | B | 66 | C |
| 6 | 100 | A | 100 | A | 100 | A | 100 | A | 100 | A | 100 | A | 88 | A | 75 | B |
| 7 | 100 | A | 100 | A | 100 | A | 100 | A | 100 | A | 100 | A | 100 | A | 92 | A |
| 8 | 100 | A | 87 | A | 75 | B | 58 | B | 45 | C | 40 | D | 40 | D | 35 | E |
| 9 | 100 | A | 100 | A | 95 | A | 92 | A | 88 | A | 82 | B | 69 | C | 52 | D |
| 10 | 100 | A | 100 | A | 100 | A | 100 | A | 95 | A | 90 | A | 78 | B | 64 | C |
| 11 | 100 | A | 100 | A | 95 | A | 86 | A | 80 | B | 74 | C | 72 | C | 70 | C |
| 12 | 100 | A | 100 | A | 97 | A | 92 | A | 88 | A | 85 | B | 83 | B | 80 | B |
| 13 | 100 | A | 100 | A | 100 | A | 95 | A | 91 | A | 90 | A | 85 | B | 82 | B |
| 14 | 100 | A | 100 | A | 100 | A | 95 | A | 90 | A | 87 | B | 80 | B | 72 | B |
| 15 | 100 | A | 100 | A | 100 | A | 100 | A | 100 | A | 100 | A | 95 | A | 92 | A |
| 16 | 100 | A | 100 | A | 100 | A | 100 | A | 100 | A | 100 | A | 100 | A | 94 | A |
| 17 | 100 | A | 100 | A | 100 | A | 96 | A | 90 | A | 85 | B | 85 | B | 82 | B |
| 18 | 100 | A | 100 | A | 100 | A | 100 | A | 100 | A | 100 | A | 100 | A | 100 | A |
| 19 | 100 | A | 100 | A | 100 | A | 100 | A | 100 | A | 100 | A | 100 | A | 100 | A |

In general, the addition of the composition comprising the compound of general formula (I), surprisingly, significantly increased the control efficiency of the herbicides at 42, 49, and 56 days after the treatments were applied. Also, overall, increasing the tested dosage of the adjuvant resulted in significant increase of the weed complex control.

Thus, the agrochemical formulation comprising the ethoxylated polyol composition according to the present invention enables greater efficiency in weed control by increasing the residual effect of pre-emergent herbicides.

The examples described herein demonstrate the advantages of agrochemical composition and formulation comprising the compound of general formula (I) in relation to the increased availability of water and nutrients for plants and seeds, to the positive effects on their development, on germination rate, water infiltration time in water repellent soil, and on synergism with pesticides in pest control.

1. A composition characterized by comprising:
a compound of general formula (I)
(R¹—O—R)ₘ (I)
wherein the compound of formula (I) is a polyol derivative having covalent bonds between the R¹ radicals;
where: m is a number ranging from 3 to 6,
R¹ is a C₁ alkyl radical,
each R is independently H or an oxyethylene group represented by [(C₂H₄O)ₙ—R²), where at least one R is [(C₂H₄O)ₙ—R²],
R² is independently a hydrogen or a C₁₋₄ alkyl chain,
each n can be the same or different and is a number ranging from 1 to 18, and a sum of all n present in the compound of general formula (I) is a number that ranges from 1 to 108,
wherein the compound of general formula (I) is a polyol derivative with m being 3 or 6 and all R being [(C₂H₄O)ₙR²], where R² is hydrogen, n is a number between 1 and 18 and, where:
when m is 3, the sum of all n present in the compound of general formula (I) is a number between 3 and 50, and
when m is 6, the sum of all n present in the compound of general formula (I) is a number between 6 and 54,
wherein the composition comprises:
i) from 8 to 60% by weight of the compound of general formula (I),
ii) from 5% to 45% by weight of at least one surfactant,
iii) from 0% to 10% by weight of at least one unmodified polyol, and
iv) water in q.s. (enough quantity for) 100% by weight, based on the total weight of the composition,
wherein the at least one unmodified polyol is selected from a group comprising: 1,2,3-Propanetriol, 1,2,3,4-Butanetetrol, 1,2,3,4,5-Pentanepentol, or 1,2,3,4,5,6-Hexanehexol.

2. (canceled)

3. (canceled)

4. (canceled)

5. (canceled)

6. (canceled)

7. The composition according to claim 1, characterized in that the at least one surfactant is selected from ethoxylated alkyl ethers, phosphated ethoxylated alkyl ethers, ethoxylated alkyl etheramines, alkyl polyglucosides, ethoxylated alkyl polyglucosides, ethoxylated imidazolines, polysiloxane derivatives, alkyl dimethyl amine oxides, alkyl dimethyl betaines, trialkyl ammonium propanoates, alkyl amido propyl amines, ethoxylated alkyl amines, ethoxylated amidoamines, alkylene oxide block or random copolymer, sorbitan esters, or polysorbates.

8. (canceled)

9. An agrochemical, characterized in that it comprises the composition as defined in claim 1, and at least one active ingredient having pesticidal or plant enhancing action, via proportion of water in soil, selected from a group comprising herbicides, insecticides, fungicides, and nematicides.

10. The agrochemical according to claim 9, characterized in that the at least one active ingredient is an herbicide, and wherein the herbicide comprises at least one of: acetamides, arylaminopropionic acid, benzoic acid, chlorocarbonic acid, phenoxycarboxylic acid, phosphinic acid, quinolinecarboxylic acid, amides, aryloxyphenoxypropionates (FOPs), arylpicolinate, benzamides, benzofurans, benzothiadiazinones, bipyridyliums, carbamates, cyclohexanediones (DIMs), chloroacetamides (V1), chloroacetamides (V2), chloroacetamides (V3), diphenyl ethers, dinitroanilines, dinitrophenols, phenylcarbamates, phenylpyrazoles, phenylpyrazolines (DENs), phenyl-pyridazines, phosphoroamidates, phosphorodithioates, semicarbazone phthalamates, glycines, imidazolinones, long chain fatty acid inhibitor, isoxazoles, isoxazolidinones, N-phenylphthalimides, nitriles, organoarsenicals, oxadiazoles, oxazolidinedinones, oxiacetamides, pyrazoles, pyrazoliums, pyridazinones, pyridines, pyridinecarboxamides, pyrimidindiones, pyrimidinyl (thio) benzoates, sulfonylaminocarbonyl-triazolinones, sulfonylureas, tetrazolinones, thiadiazoles, thiocarbamates, triazines, triazinones, triazoles, triazolinones, triazolocarboxamides, triazolopyrimidines, tricetones, uracils, or ureas.

11. The agrochemical according to claim 9, characterized in that the at least one active ingredient is an insecticide, and wherein the insecticide comprises at least one of: a group of METI acaricides and insecticides, acequinocyl, aliphatic halogenated, amitraz, nereistoxin analogues, juvenile hormone analogues, avermectins, milbemicins, azadirachtin, Bacillus sp. and produced insecticidal proteins, benzoylurea, benzoximate, bifenazate, borates, borax, bromopropylate, buprofezin, butenolides, lime sulphur, carbamates, carboxanilides, cyanides, cyclodienes, cyromazine, clofentezine, diflovidazin, hexithiazoxy, chlorfenapyr, dinitrophenol, sulfluramid, chloropicrin, DDT, methoxychlor, tetronic and tetramic acid derivatives, betaketonitrile derivatives, azomethine pyridine derivatives, diacylhydrazines, diafenthiuron, diamides, dicofol, spinosynes, ethoxazole, phenylpyrazoles (fiproles), fenoxycarb, rotenoid-type saponin flavones, flonicamide, fluacripyrim, fluorides, phosphides, methyl isothiocyanate generators, hydramethylnone, inorganic, inorganic phosphine precursor, methyl isothiocyanate precursor, organophosphate, mesoionics, neonicotinoids, nicotine, organotins, organophosphates, oxadiazines, GS-omega/kappa HXTXHv1a peptide, pyrazole, pyrethroids and pyrethrins, pyridalyl, pyriproxyfen, propargite, quinomethionate, rotenone, semicarbazones, fluoroaliphatic sulfonamide, sulfoxaflor, or tetradifone.

12. The agrochemical according to claim 9, characterized in that the at least one active ingredient is a fungicide, and wherein the fungicide comprises at least one of: 1,2,4-thiadiazole, 2,6-dinitro-aniline, 4-quinolyl-acetate, carboxylic acid, enopyranuronic acid, phthalamic acid, acylalanine, allylamine, mandelic acid amides, cinnamic acid amides, aminocyanoacrylate, amino-pyrazolinone, anilinopyrimidine, anthraquinone, aryloxyquinoline, Bacillus sp. and the produced fungicide lipopeptides, benzene sulfonamide, benzylcarbamate, benzimidazole, benzisothiazole, benzophenone, benzoylpyridine, benzothiadiazole BTH, benzotriazine, butyrolactone, carbamate, carboxamide, cyanoacetamide-oxime, cyanoimidazole, cyano-methylene-thiazolidine, cyclopropane carboxamide, chloronitrile, triphenyltin compounds, dicarboximide, dihydro-dioxazine, dinitrophenyl crotonate, dithiocarbamates and relative, dithiolane, spirocetal-amine, ethyl phosphonate, ethylamino-thiazol-carboxamide, phenyl-acetamide, phenyl-benzamide, phenyl-oxo-ethyl thiophene amide, phenylpyrrole, phenylurea, phosphonate, phosphorothiolate, phthalimide, furan-carboxamide, guanidine, aromatic hydrocarbon, terpene hydrocarbons and terpene alcohols, hydroxy-(2-amino-) pyrimidine, hydroxyaniline, imidazole, imidazolinone, inorganic (copper), inorganic (sulfur), isobenzofuranone, isothiazolone, isoxazole, maleimide, methoxy-acetamide, methoxy-acrylate, methoxy-carbamate, Reynoutria sp. extract, morpholine, N-phenyl carbamate, N-methoxy-(phenyl-ethyl)-pyrazol-carboxamide, pyrimidine peptidyl nucleoside, oxatin-carboxamide, oxazolidine-dione, oxazolidinone, oximine-acetamide, oximine-acetate, piperazine, piperidine, piperidinyl-thiazole-isoxazoline, pyrazole-4-carboxamide, pyrazole-5-carboxamide, pyridazinone, pyridine, pyridine-carboxamide, pyridinyl-ethyl benzamide, pyridinylmethyl-benzamide, pyrimidine, pyrimidinamine, pyrimidinone-hydrazone, pyrrolo-quinolinone, polypeptide (lectin), polysaccharide, propionamide, quinazolinone, quinoxaline, sulfamoyl-triazole, sulfonamide, tetrazoyloyloxime, thiadiazol-carboxamide, thiazol-carboxamide, thiocarbamate, thiophanate, thiophene-carboxamide, toluamide, triazine, triazole, triazolintione, triazolobenzo-thiazole, triazole-pyrimidylamine, Trichoderma sp. and produced fungicidal metabolites, trifluoroethylcarbamate, or valinamide carbamate.

13. The agrochemical according to claim 9, characterized in that the at least one active ingredient is a nematicide comprising at least one of: a halogenated aliphatic compound, avermectin, benzamides, plant extract, fluoroalkenyle (-thiother), methyl isothiocyanate precursor, benzofuranyl methylcarbamate, or organophosphate.

14. The agrochemical according claim 9, characterized in that it comprises at least one additional ingredient selected from nutrients, microorganisms, and growth regulating/bio-stimulant compounds.

15. The agrochemical according to claim 14, characterized in that the nutrients are macronutrients selected of N, P, K, S, Ca, Mg or mixtures thereof, and/or micronutrients selected of Fe, Zn, Mn, Cu, Ni, Cl, Mo, B, Si, Se, Al, Co, V, or Na.

16. The agrochemical according to claim 14, characterized in that the microorganisms are Amblyseius sp., Azozpirillum sp., Bacillus sp., Baculovirus sp., Beauveria sp., Cotesia sp., Cryptolaemus sp., Deladenus sp., Heterorhabditis sp., Isaria sp., Metarhizium sp., Neoseiulus sp., Orius sp., Paecilomyces sp., Pasteuria sp., Phytoseiulus sp., Pseudomonas sp., Stratiolaelaps sp., Telenomus sp., Trichoderma sp., or Trichogramma sp.

17. The agrochemical formulation according to claim 14, characterized in that the growth regulator/bio-stimulant compounds comprise one or more of the following compounds: dioxocyclohexanecarboxylic acid, indolalcanoic acid, aliphatic alcohol, quaternary ammonium, carbimide, carboxanilide, cycloalkene, cyclohexadione, cytokinin, dinitroaniline, ethylene inhibitor, ethylene precursor, gibberellin, pyridazinadione, sesquiterpenes, triazole, benzothiadiazol, nitric oxide, methyl salicylate, methyl jasmonate, proteins, polypeptides, polyamines, algae extracts, fulvic acid, humic acid, or plant growth promoting rhizobacteria.

18. The agrochemical formulation according to claim 9, characterized in that the composition is for use in agriculture.

19. A method for increasing an availability of water and nutrients to plants and seeds characterized by comprising the steps of:
providing a composition comprising a compound of general formula (I)
(R¹—O—R)ₘ (I)
where: m is a number ranging from 3 to 6,
R¹ is a C₁ alkyl radical,
each R is independently H or an oxyethylene group represented by [(C₂H₄O)ₙ—R²), where at least one R is [(C₂H₄O)ₙ—R²],
R² is independently a hydrogen or a C₁₋₄ alkyl chain,
each n can be the same or different and is a number ranging from 1 to 18, and a sum of all n present in the compound of general formula (I) is a number ranging from 1 to 108, and
applying the composition to a seed, soil, liquid medium, or inert substrate.

20. The method according to claim 19, characterized in that the step of applying the composition occurs by means of seed treatment, by gravity irrigation system, by sprinkler, by drip, by infiltration, by spraying, by central pivot irrigation, by hydroponics system, or by submersion, wherein the composition is applied in concentration from 1 ppm to 10,000 ppm.

21. The method according to claim 19, characterized in that the composition is applied to agricultural plantations, ornamental lawn/turf areas, or forests.

22. The method according to claim 19, characterized in that the compound of general formula (I) is a polyol derivative with m being 3 or 6 and all R being [(C₂H₄O)ₙ—R²], where R² is hydrogen, n is a number between 1 and 18 and, where:
when m is 3, a sum of all n present in the compound of general formula (I) is a number between 3 and 50, and
when m is 6, a sum of all n present in the compound of general formula (I) is a number between 6 and 54.

23. The method according to claim 19, characterized in that the composition comprises:
i) from 5 to 100% by weight of the compound of general formula (I),
ii) from 0 to 60% by weight of at least one surfactant,
iii) from 0 to 40% by weight of at least one unmodified polyol, and
iv) water to q.s. (enough quantity for) 100% by weight, based on the total weight of the composition.

24. The method according to claim 23, characterized in that the composition comprises:
i) from 5 to 80% by weight of the compound of general formula (I),
ii) from 0 to 45% by weight of at least one surfactant,
iii) from 0 to 30% by weight of at least one unmodified polyol, and
iv) water to q.s. (enough quantity for) 100% by weight, based on the total weight of the composition.

25. The method according to claim 23, characterized in that the composition comprises:
i) from 8 to 60% by weight of the compound of general formula (I),
ii) from 5% to 45% by weight of at least one surfactant,
iii) from 0% to 10% by weight of at least one unmodified polyol, and
iv) water to q.s. (enough quantity for) 100% by weight, based on the total weight of the composition.

26. The method according to claim 23, characterized in that the at least one unmodified polyol is selected from a group comprising: 1,2,3-Propanetriol, 1,2,3,4-Butanetetrol, 1,2,3,4,5-Pentanepentol, or 1,2,3,4,5,6-Hexanehexol.

27. The method according to claim 23, characterized in that the at least one surfactant is selected from ethoxylated alkyl ethers, phosphated ethoxylated alkyl ethers, ethoxylated alkyl etheramines, alkyl polyglucosides, ethoxylated alkyl polyglucosides, ethoxylated imidazolines, polysiloxane derivatives, alkyl dimethyl amine oxides, alkyl dimethyl betaines, trialkyl ammonium propanoates, alkyl amido propyl amines, ethoxylated alkyl amines, ethoxylated amidoamines, alkylene oxide block or random copolymer, sorbitan esters, or polysorbates.

28. A method for improving pest control in plants and seeds comprising the steps of:
providing an agrochemical formulation as defined in claim 9; and
applying the agrochemical formulation to a seed, soil, liquid medium, or inert substrate.

29. The method according to claim 28, characterized in that the step of applying the formulation occurs by means of seed treatment, by gravity irrigation system, by sprinkler, by drip, by infiltration, by spraying, by central pivot irrigation, by hydroponic system, or by submergence, wherein the formulation is applied in concentrations from about 1 ppm to about 10,000 ppm.

30. The method according to claim 28, characterized in that the composition is applied to agricultural plantations, ornamental lawn/turf areas, or forests.
